(19) United States
(12) Patent Application Publication
(10) Pub. No.: US 2026/0134536 A1
(43) Pub. Date: May 14, 2026

(54) METHOD OF AUTOMATIC SEGMENTATION FOR VOLUME SWEEP IMAGING PROTOCOL IN LUNGS

(71) Applicants: UNIVERSITY OF ROCHESTER, Rochester, NY (US); CATHOLIC UNIVERSITY OF PERU, San Miguel (PE)

(72) Inventors: Benjamin CASTANEDA APHAN, Rochester, NY (US); Emilio J. OCHOA ALVA, Rochester, NY (US); Thomas J. MARINI, Rochester, NY (US); Luis C. REVILLA DOMINGUEZ, San Miguel (PE); Gabriel O. DURAN RUIZ, San Miguel (PE); Stefano E. ROMERO GUTIERREZ, San Miguel (PE)

(21) Appl. No.: 19/384,503

(22) Filed: Nov. 10, 2025

Related U.S. Application Data
(60) Provisional application No. 63/718,977, filed on Nov. 11, 2024.

(51) Int. Cl. G06T 7/00; G06T 7/10; G16H 30/40

(52) U.S. Cl. CPC G06T 7/0012; G06T 7/10; G16H 30/40; G06T 2207/10132; G06T 2207/20084; G06T 2207/30061

(57) ABSTRACT

A deep learning system that detects and counts A-lines in lung images obtained with the VSI protocol, allowing untrained personnel to perform scans. By segmenting and counting A-lines, the system distinguishes between healthy lungs and conditions such as pneumonia or edema, without a specialist intervention. The Attention U-Net architecture improves accuracy, and A-line counting provides quantitative data for diagnostics. This approach aims to expand access to diagnostics, specially in resource-limited areas, offering a scalable solution for early detection of lung disease.

This application claims priority of U.S. Provisional Application No. 63/718,977, filed on Nov. 11, 2024, which is hereby incorporated by reference.

### FIELD

This application relates to the field of radiology and, in particular, the generation and analysis of ultrasound images.

### BACKGROUND

The lack of access to radiologists and sonographers in rural areas presents a barrier to timely and accurate diagnosis of pulmonary diseases. Point-of-Care Ultrasound (POCUS) has emerged as a practical, cost-effective tool for lung imaging in these settings. Although POCUS is portable and inexpensive, it requires expert interpretation. Volume Sweep Imaging (VSI) protocol allows naïve operators to acquire biomedical ultrasound images with minimal training. However, there still exists a need for rapid and accurate analysis of the ultrasound images at low cost.

### SUMMARY

The present application provides a system that can triage between normal and abnormal lungs based on the use of a deep learning algorithm to segment Pleural and A lines from videos (cine loop/sweeps) within a Lung Volume Sweep Imaging ultrasound acquisition. An image processing algorithm quantifies the A-lines per frame to distinguish normal from abnormal studies. The significant group differences and the model's cross-site robustness support its implementation in several settings. Beyond technical performance, this approach enables adequate clinical triage by allowing naïve operators to perform standardized scans with handheld devices and receive automated feedback. In rural clinics, outpatient facilities, and large-scale screening campaigns, this system could guide appropriate referrals and accelerate patient management. By integrating artificial intelligence (AI)-based interpretation with the standardized VSI protocol, the method provides a scalable pathway to equitable pulmonary imaging and improved access to early diagnosis and treatment in underserved populations, outpatient settings and triage in health campaigns or hospitals.

One aspect of the application relates to a method of automatic segmentation for volume sweep imaging, comprising the steps of: gathering lung ultrasound images using the volume sweep imaging protocol; cropping images to focus on relevant lung areas; manually segmenting A-lines and relevant structures; assigning RGB colors to ground truth for consistent identification; selecting frames with masks to focus on informative images for analysis; applying an algorithm to replicate manual segmentation for scalability; implementing an algorithm to count A-lines, wherein the algorithm distinguishes between normal and abnormal scans.

Another aspect of the present application relates to a method of analyzing ultrasound images with an image processing system, comprising the steps of: gathering lung ultrasound images of a subject using a VSI protocol; cropping, by the image processing program, the gathered images to generate frames focusing on relevant lung areas; segmenting, by the image processing program, A-lines and other relevant structures from the frames; and quantifying, by the image processing program, the number of A-lines in in each frame.

Another aspect of the present application relates to a method of training an image processing program, comprising the steps of: (a) acquiring lung ultrasound images of a subject, wherein the lung ultrasound images are generated using a volume sweep imaging (VSI) protocol; (b) cropping the acquired lung ultrasound images to generate frames focusing on relevant lung areas; (c) segmenting, by a human medical professional, A-lines and relevant structures in the cropped lung ultrasound images to produce manual segmentations as ground truth; (d) assigning red-green-blue (RGB) colors to the ground truth to produce ground truth masks; (e) selecting frames with masks to focus on informative images for analysis; (f) applying a segmentation algorithm to the acquired lung ultrasound images to replicate the manual segmentations; and (g) repeating steps (a)-(f) until a desired hit-and-miss ratio (HMR) is achieved.

Another aspect of the present application relates to an image processing system, comprising: one or more computer processors; and one or more tangible computer readable media accessible by the one or more computer processors, wherein the one or more tangible computer readable media comprise instructions that, when executed by the one or more processors, cause the one or more processors to perform: (a) receiving, via a user interface of an application executing on one or more computer processors, lung ultrasound images of a subject, wherein the lung ultrasound images are generated using a VSI protocol; (b) cropping, via the one or more computer processors, the gathered images to generate frames focusing on relevant lung areas; and (c) segmenting, via the one or more computer processors, A-lines and other relevant structures from the frames.

Another aspect of the present application relates to a tangible non-transitory computer readable storage medium, comprising instructions that, when executed by a computer processor, cause the processor to: (a) receiving, via a user interface of an application executing on one or more computer processors, lung ultrasound images of a subject, wherein the lung ultrasound images are generated using a VSI protocol; (b) cropping, via the one or more computer processors, the gathered images to generate frames focusing on relevant lung areas; and (c) segmenting, via the one or more computer processors, A-lines and other relevant structures from the frames.

These and other aspects, objects, features, and advantages of the example embodiments will become apparent to those having ordinary skill in the art upon consideration of the following detailed description of example embodiments.

An understanding of the features and advantages of the present application will be obtained by reference to the following detailed description that sets forth illustrative embodiments, in which the principles of the application may be utilized, and the accompanying drawings. The drawings herein are for illustrative purposes only.

### BRIEF DESCRIPTION OF THE DRAWINGS

FIG. 1 shows a VSI protocol applied to lung ultrasound.

FIG. 2 shows automatic segmentation and quantification of A-line system workflow. Non-physician health personnel perform the standardized VSI Lung acquisition protocol using a handheld probe at the health center. The acquired clips are stored locally or uploaded to a secure cloud server for automated A-line segmentation and quantification. The results can be reviewed on-site or transmitted to a remote radiologist for validation and diagnostic support.

FIG. 3 shows an exemplary training workflow for the automatic segmentation and A-line counting with VSI protocol in Lungs. Steps 1 and 3 were performed by physicians, while steps 2, 4-6 were automated.

FIG. 4 shows exemplary Attention U-Net architecture

FIG. 5 shows exemplary details of automated interaction between an operator and the image processing algorithm. It shows the different parts of the algorithm and that the algorithm can be performed locally in a tablet or remotely in a cloud server.

FIG. 6 shows a comparison between a brightness mode (B-mode) ultrasound image (left), ground-truth segmentation (center), and AI label predictions (right) for two different studies. Red: pleural line, green: strong A-line, blue: weak A-line. B-mode image.

FIG. 7 shows a boxplot of relative A-lines per frame according to abnormal vs. normal lung diagnosis.

FIG. 8 shows the average A-lines predicted by AI model (normal vs abnormal studies) from Peru. Normal cases consistently showed higher A-line counts than abnormal ones across all evaluated models, reflecting the expected decrease of reverberation artifacts in pathological lungs. Median A-line values were approximately 1.8 (abnormal) and 2.3 (normal) for the Attention U-Net, 1.2 (abnormal) and 2.4 (normal) for the Multi Attention U-Net, and 1.0 (abnormal) and 2.5 (normal) for the U-Net.

FIG. 9 shows the average A-lines predicted by AI model (normal vs abnormal studies) from Rochester. The distribution of A-line counts per patient shows a clear separation between normal and abnormal studies. Normal cases presented higher predicted A-line values, with a median of approximately 2.55, compared to 1.47 in abnormal cases.

### DETAILED DESCRIPTION

Reference will be made in detail to certain aspects and exemplary embodiments of the application, illustrating examples in the accompanying structures and figures. The aspects of the application will be described in conjunction with the exemplary embodiments, including methods, materials and examples, such description is non-limiting and the scope of the application is intended to encompass all equivalents, alternatives, and modifications, either generally known, or incorporated here. Unless otherwise defined, all technical and scientific terms used herein have the same meaning as commonly understood by one of ordinary skill in the art to which this application belongs. One of skill in the art will recognize many techniques and materials similar or equivalent to those described here, which could be used in the practice of the aspects and embodiments of the present application. The described aspects and embodiments of the application are not limited to the methods and materials described.

As used in this specification and the appended claims, the singular forms “a,” “an” and “the” include plural referents unless the content clearly dictates otherwise.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to “the value,” greater than or equal to the value” and possible ranges between values are also disclosed, as appropriately understood by the skilled artisan. For example, if the value “10” is disclosed the “less than or equal to 10” as well as “greater than or equal to 10” is also disclosed.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

In the following description, for purposes of explanation, numerous specific details are set forth in order to provide a thorough understanding of the present invention. It will be evident, however, to one skilled in the art that the present invention may be practiced without these specific details.

### I. Definitions

As used herein, the term “A-lines” refers to horizontal, bright, hyperechoic lines that appear on a lung ultrasound, indicating well-aerated lung tissue. A-lines are a type of reverberation artifact caused by the ultrasound beam reflecting off the air-filled lung and bouncing between the pleura and the transducer. In some embodiments, the A-lines are arbitrarily designated as “strong A-lines” or “weaker A-lines” based on the intensity of the respective A-lines.

As used herein, the term “pleural lines” refers to bright, hyperechoic lines that represent the interface between the chest wall and the lungs in lung ultrasound images. The appearance, movement, and relationship with other artifacts are crucial for diagnosing many lung conditions.

As used herein, the term “volume sweep imaging (VSI)” refers to an ultrasound technique that uses a standardized protocol of probe movements, or “sweeps” and “arcs,” to capture images of a target area, such as lung, breast, liver, bladder and uterus. It is designed to be performed by operators with minimal training by following external anatomical landmarks, and the resulting video clips are saved for later interpretation by an expert or a computer program, such as the AI-assisted image process program of the present application. This approach increases the accessibility of ultrasound diagnostics, particularly in remote or underserved areas.

As used herein, the term “cine loop” refers to a recorded video of a series of images that allow for review of motion and movement. In some embodiments, the images are medical images. In some embodiments, the medical images are ultrasound images. In some embodiments, a cine loop is a recording, which can be digital or analog, of a real-time or dynamic process. A cine loop can be saved, replayed, and scrolled through (or scanned) to find a frame, or frames, allowing for analysis or diagnosis.

The term “computer,” as used herein, refers to a machine, apparatus, or device that is capable of accepting and performing logic operations from software code. The term “application”, “software”, “software code,” “computer software” or “computer program,” refers to any set of instructions operable to cause a computer to perform an operation. Software code may be operated on by a “rules engine” or processor. Thus, the methods and systems of the present invention may be performed by a computer or computing device having a processor based on instructions received by computer applications and software.

The term “electronic device,” as used herein, refers to a type of computer comprising circuitry and configured to generally perform functions such as recording audio, photos, and videos; displaying or reproducing audio, photos, and videos; storing, retrieving, or manipulation of electronic data; providing electrical communications and network connectivity; or any other similar function. Non-limiting examples of electronic devices include: personal computers (PCs), workstations, laptops, tablet PCs including the iPad, cell phones including iOS phones made by Apple Inc., Android OS phones, Microsoft OS phones, Blackberry phones, digital music players, or any electronic device capable of running computer software and displaying information to a user, memory cards, other memory storage devices, digital cameras, external battery packs, external charging devices, and the like. Certain types of electronic devices which are portable and easily carried by a person from one location to another may sometimes be referred to as a “portable electronic device” or “portable device”. Some non-limiting examples of portable devices include: cell phones, smartphones, tablet computers, laptop computers, wearable computers such as Apple Watch, other smartwatches, Fitbit, other wearable fitness trackers, Google Glasses, and the like.

The term “client device” as used herein, refers to a type of computer or computing device comprising circuitry and configured to generally perform functions such as recording audio, photos, and videos; displaying or reproducing audio, photos, and videos; storing, retrieving, or manipulation of electronic data; providing electrical communications and network connectivity; or any other similar function. Non-limiting examples of client devices include: personal computers (PCs), workstations, laptops, tablet PCs including the iPad, cell phones including iOS phones made by Apple Inc., Android OS phones, Microsoft OS phones, Blackberry phones, Apple iPads, Anota digital pens, digital music players, or any electronic device capable of running computer software and displaying information to a user, memory cards, other memory storage devices, digital cameras, external battery packs, external charging devices, and the like. Certain types of electronic devices which are portable and easily carried by a person from one location to another may sometimes be referred to as a “portable electronic device” or “portable device”. Some non-limiting examples of portable devices include: cell phones, smartphones, tablet computers, laptop computers, tablets, digital pens, wearable computers such as Apple Watch, other smartwatches, Fitbit, other wearable fitness trackers, Google Glasses, and the like.

The term “computer readable medium” as used herein, refers to any medium that participates in providing instructions to the processor for execution. A computer readable medium may take many forms, including but not limited to, non-volatile media, volatile media, and transmission media. Non-volatile media includes, for example, optical, magnetic disks, and magneto-optical disks, such as the hard disk or the removable media drive. Volatile media includes dynamic memory, such as the main memory. Transmission media includes coaxial cables, copper wire and fiber optics, including the wires that make up the bus. Transmission media may also take the form of acoustic or light waves, such as those generated during radio wave and infrared data communications.

As used herein the term “data network” or “network” refers to an infrastructure capable of connecting two or more computers such as client devices either using wires or wirelessly allowing them to transmit and receive data. Non-limiting examples of data networks may include the internet or wireless networks or (i.e. a “wireless network”) which may include Wifi and cellular networks. For example, a network may include a local area network (LAN), a wide area network (WAN) (e.g., the Internet), a mobile relay network, a metropolitan area network (MAN), an ad hoc network, a telephone network (e.g., a Public Switched Telephone Network (PSTN)), a cellular network, a Zigby network, or a voice-over-IP (VoIP) network.

As used herein, the term “database” generally refers to a digital collection of data or information. The present invention uses novel methods and processes to store, link, and modify information such digital images and videos and user profile information. For the purposes of the present disclosure, a database may be stored on a remote server and accessed by a client device through the internet (i.e., the database is in the cloud) or alternatively in some embodiments the database may be stored on the client device or remote computer itself (i.e., local storage). A “data store” as used herein may contain or comprise a database (i.e. information and data from a database may be recorded into a medium on a data store).

As used herein, the term “cloud” refers to “cloud computing,” such as on-demand computing services like servers, storage, software, and analytics over the internet. As an alternative to storing data and running programs on a local computer, resources are accessed from remote servers hosted in data centers.

As used herein, the term “AI-assisted” or “AI-based” refer to processes using artificial intelligence (AI) as a tool to assist an operator in performing a task or function or in the analysis of data. For example, in the present application an algorithm, image processing program, image processing system may be AI-assisted or AI-based.

### II. Method for Analyzing Ultrasound Images Using an Image Processing Program

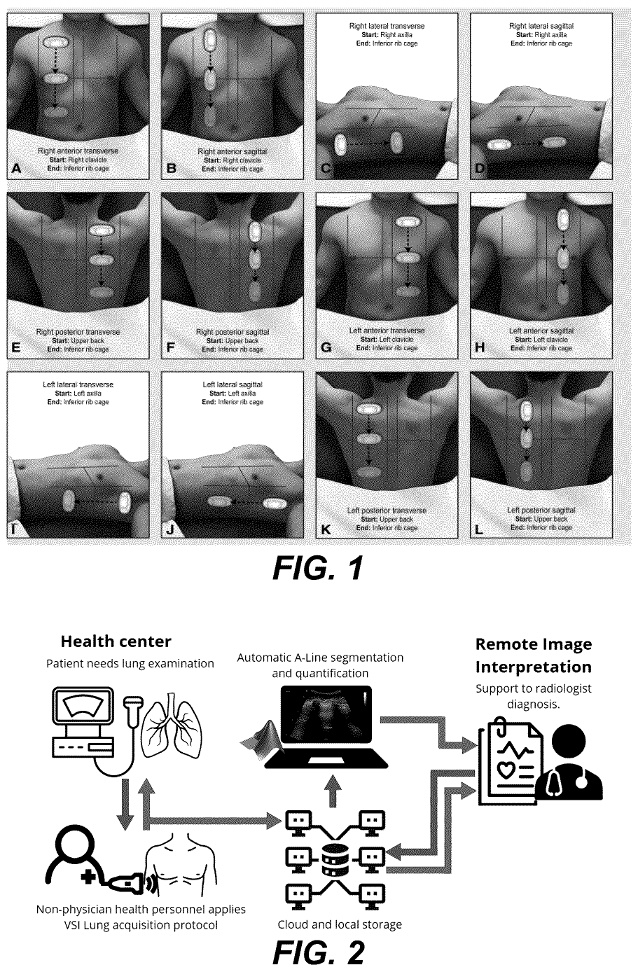

One aspect of the present application relates to a method for analyzing ultrasound images of the lungs using an image processing program. FIG. 1 shows an exemplary VSI protocol of sweeps of the lung. In lung ultrasound, horizontal reverberation artifacts known as A-lines are typically associated with aerated parenchyma, whereas reduced A-line density may reflect pathology (pulmonary edema, pneumonia and pulmonary effusion). The present application provides an image processing method that segments pleural and A-lines and quantifies A-lines per frame to support screening and triage performed by non-expert operators following a VSI Lung protocol.

In some embodiments, the imaging processing program is an AI-assisted imaging processing program.

In normal aerated lung, hyperechoic, horizontal lines (A-lines) arising at regular intervals from the pleural line can be seen in ultrasound images. These are reverberation artifacts that arise when the ultrasound beam reflects off of the pleura and, instead of entering the probe, partially reflects off of the probe face back to the pleura again before getting back to the ultrasound probe. This double-length pathway is interpreted and displayed as if the source of the echo lies at two times the distance between pleura and skin because, the distance at which a particular structure is displayed on the ultrasound screen depends on how long the echoes returning from that structure take to reach the probe. Multiple reverberations result in multiple A-lines, at multiples of the pleural depth. In short, if A-lines are present, the lungs are filled with air.

In some embodiments, the method of the present application comprises the steps of gathering lung ultrasound images of a subject using a VSI protocol; cropping, by the image processing program, the gathered images to generate frames focusing on relevant lung areas; segmenting, by the image processing program, A-lines and other relevant structures from the frames, quantifying, by the Image processing program, the number of A-lines and generating, by the image processing program, an ultrasound result based on the quantification of the A-lines.

In some embodiments, the method further comprises the step of reviewing, by a trained expert, the ultrasound result generated by the by the image processing program. In some embodiments, the reviewing step is performed by a radiologist at a remote location from the site of VSI.

In some embodiments, the lung ultrasound images are gathered by a person trained to perform VSI. In some embodiments, the gathering step is performed by a non-physician health personnel. In some embodiments, the lung ultrasound images of a subject are sagittal images. In some embodiments, the lung ultrasound images of a subject are transverse images.

In some embodiments, the acquiring step comprises acquiring multi-modal input images through an encoder. In some embodiments, encoded multi-modal input images are processed by a decoder before becoming segmentation output images.

In some embodiments, the segmenting step is performed by an AI-assisted image processing program using a segmentation algorithm. In some embodiments, the segmentation algorithm identifies the pleural lines, strong A-lines and weak A-lines in the cropped images. The image processing program then generates and applies RGB color masks to the segmented images. In some embodiments, red is assigned to pleural lines, green is assigned to strong A-lines, and blue is assigned to weak A-lines in a RGB color mask.

In some embodiments, the quantifying step is executed by an A-line counter algorithm operating on both the ground truth and predicted RGB color masks. Connected components are computed using Explicar 8 connectivity for pixel and region discrimination.

In some embodiments, the method of the present application comprises the steps of gathering lung ultrasound images of a subject using a VSI protocol; cropping, by the image processing program, the gathered images to generate frames focusing on relevant lung areas; segmenting, by the image processing program, A-lines and other relevant structures from the frames, quantifying, by the Image processing program, the number of A-lines to produce an average A-lines per frame (ALF); and generating, by the image processing program, an ultrasound result based on the ALF of the lung ultrasound images of the subject.

In some embodiments, the ultrasound result of the subject is either abnormal or normal. In some embodiments, an abnormal ALF has a value equal to or below 0.6. In some embodiments, an abnormal ALF has a value between 0 and 0.6, between 0.1 and 0.6, or between 0.2 and 0.6. In some embodiments, an abnormal ALF has a value between 0.3 and 0.6. In some embodiments, a normal ALF has a value equal to or above 0.7. In some embodiments, a normal ALF has a value between 0.7 and 1.0. In some embodiments, an ALF having a value between 0.6 and 0.7 is identified as borderline. In general, ALF values results can be used by a practitioner to suggest the appropriate follow up in the care of the patient. For example, a patient with an abnormal ALF value is a strong candidate for follow-up care, while a patient with a borderline ALF value should consider follow-up evaluation or care.

In some embodiments, the method further comprises a step of using reinforcement learning to enable the image processing program to adapt to new data based on input on segmentations, wherein clinicians provide input on segmentations.

The image processing program and algorithms of the present application allow for a non-expert to use a point of care ultrasound device to perform a lung VSI protocol and screen the lung of the patient for abnormalities. The program and algorithms of the present application have been tested to identify patients with a normal vs. abnormal lung. The program and algorithms of the present application can also be used to identify abnormal indications for a particular anatomical region in the lung (i.e. right lung, front side, upper part). In addition, the program and algorithms of the present application can be used to characterize abnormalities in the presence of A-lines, related not only to the presence of fluid in the lung, but also consolidations.

In the method of the present application, the POCUS-based triage can be performed by an ultrasound-naïve operator, reducing dependence on expert operators for both image acquisition and interpretation. Using a handheld probe and a mobile interface, the operator may perform a lung VSI acquisition. The operator would acquire cine loops, run the proposed algorithm locally or via cloud processing, and receive automated feedback regarding the likelihood of normal versus abnormal lung patterns. This workflow supports a task-shifting paradigm in which primary-care or community health personnel can perform the initial screening and then refer suspicious cases to higher-level facilities for confirmatory imaging or specialist review.

Furthermore, the framework supports integration with teleultrasound systems, enabling remote verification and annotation by radiologists. Incorporating active learning or reinforcement learning mechanisms in future iterations could allow the image processing program and algorithms of the present application to continuously refine its segmentation performance using clinician feedback. Beyond pulmonary applications, the same AI-VSI pipeline could be adapted to other anatomical regions of interest, including the thyroid, breast, placenta, and fetal structures.

The technology described here can be applied in different ways. In some embodiments, a patient is in Emergency Department triage and a practitioner would run the probe over the patient and say whether the patient has abnormal lungs or not. In some embodiments, a caregiver would run the probe over a senior resident and say whether the patient has abnormal lungs (i.e. pneumonia) or not. In some embodiments, healthcare personnel in a remote outpatient setting can use this technology to screen a little child for pneumonia. In some embodiments, a patient presents at a clinic where, after running the probe over the patient, it could be said whether the patient has abnormal lungs or not.

Another aspect of the present application relates to a method for analyzing ultrasound images using an image processing program. The method comprises the steps of acquiring ultrasound images of a target organ or tissue of a subject, wherein the ultrasound images are produced using a VSI protocol, analyzing the ultrasound images using the image processing program and generating a diagnosis. In some embodiments, the method further comprises the step of confirming the diagnosis with an expert. The target area can be any organ or tissue of the subject that is amenable to ultrasound image. Examples of such organs and tissues include, but are not limited to, internal organs like the lungs, liver, pancreas, kidneys, spleen, small intestine, colon, prostate and gallbladder; reproductive organs such as the uterus and ovaries; cavities like cranial cavity and sinus cavities, and tissues like the thyroid, breast, muscles, tendons, and ligaments.

In some embodiments, the imaging procession program is AI-assisted.

### III. Method of Training the Image Processing Program

Another aspect of the present application relates to a method for training the image processing program of the present application. The method of comprises the steps of: (a) acquiring lung ultrasound images of a subject, wherein the lung ultrasound images are generated using a volume sweep imaging (VSI) protocol; (b) cropping the acquired lung ultrasound images to generate frames focusing on relevant lung areas; (c) segmenting, by a human medical professional, A-lines and relevant structures in the cropped lung ultrasound images to produce manual segmentations as ground truth; (d) assigning red-green-blue (RGB) colors to the ground truth to produce ground truth masks; (e) selecting frames with masks to focus on informative images for analysis; (f) applying an AI-assisted segmentation algorithm to the acquired lung ultrasound images to replicate the manual segmentations; (g) applying an AI-assisted counting algorithm to calculate average A-lines per frame (ALF) and produces a scan result based on ALF of the lung ultrasound images of a subject; and (h) repeating steps (a)-(g) until a desired hit-and-miss ratio (HMR) is achieved.

In some embodiments, the imaging procession program is AI-assisted.

In some embodiments, the A-lines and relevant structures comprise strong A-lines, weak A-lines and pheural lines.

In some embodiments, the AI-assisted segmentation algorithm to the acquired lung ultrasound images to replicate the manual segmentation is trained using sagittal clips from a population of patients in comparison to transverse clips.

In some embodiments, the lung ultrasound images are stored in cloud storage.

In some embodiments, the lung ultrasound images are stored in local storage.

### IV. System for Analyzing Ultrasound Images Using an Image Processing Program

Another aspect of the application relates to a system for analyzing ultrasound images using an image processing program. The system comprises one or more computer processors; and one or more tangible computer readable media accessible by the one or more computer processors, wherein the one or more tangible computer readable media comprise instructions that, when executed by the one or more processors, cause the one or more processors to perform: (a) receiving, via a user interface of an application executing on one or more computer processors, lung ultrasound images of a subject, wherein the lung ultrasound images are generated using a VSI protocol; (b) cropping, via the one or more computer processors, the gathered images to generate frames focusing on relevant lung areas; (c) segmenting, via the one or more computer processors, A-lines and other relevant structures from the frames; (d) quantifying, via the one or more computer processors, the number of A-lines in each frame to produce an average A-lines per frame (ALF); and (e) generating, via the one or more computer processors, an ultrasound scan result based on the ALF.

In some embodiments, the imaging processing program is an AI-assisted imaging processing program.

It will be appreciated that some exemplary embodiments described herein may include one or more generic or specialized processors (or “processing devices”) such as microprocessors, digital signal processors, customized processors and field programmable gate arrays (FPGAs) and unique stored program instructions (including both software and firmware) that control the one or more processors to implement, in conjunction with certain non-processor circuits, some, most, or all of the functions of the methods and/or systems described herein. Alternatively, some or all functions may be implemented by a state machine that has no stored program instructions, or in one or more application specific integrated circuits (ASICs), in which each function or some combinations of certain of the functions are implemented as custom logic. Of course, a combination of the two approaches may be used. Moreover, some exemplary embodiments may be implemented as a computer-readable storage medium having computer readable code stored thereon for programming a computer, server, appliance, device, etc. each of which may include a processor to perform methods as described and claimed herein. Examples of such computer-readable storage mediums include, but are not limited to, a hard disk, an optical storage device, a magnetic storage device, a ROM (Read Only Memory), a PROM (Programmable Read Only Memory), an EPROM (Erasable Programmable Read Only Memory), an EEPROM (Electrically Erasable Programmable Read Only Memory), a Flash memory, and the like.

Embodiments of the subject matter and the functional operations described in this specification can be implemented in digital electronic circuitry, or in computer software, firmware, or hardware, including the structures disclosed in this specification and their structural equivalents, or in combinations of one or more of them. Embodiments of the subject matter described in this specification can be implemented as one or more computer program products, i.e., one or more modules of computer program instructions encoded on a tangible program carrier for execution by, or to control the operation of, data processing apparatus. The tangible program carrier can be a propagated signal or a computer readable medium. The propagated signal is an artificially generated signal, e.g., a machine generated electrical, optical, or electromagnetic signal that is generated to encode information for transmission to suitable receiver apparatus for execution by a computer. The computer readable medium can be a machine readable storage device, a machine readable storage substrate, a memory device, a composition of matter effecting a machine readable propagated signal, or a combination of one or more of them.

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, solid state drives, or optical disks. However, a computer need not have such devices.

Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD ROM disks. The processor and the memory can be supplemented by, or incorporated in, special purpose logic circuitry.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

Embodiments of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described is this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), e.g., the Internet.

The computing system can include clients and servers. A client and server are generally remote from each other and typically interact through a communication network or the cloud. The relationship of client and server arises by virtue of computer programs running on the respective computers and having a client server relationship to each other.

Further, many embodiments are described in terms of sequences of actions to be performed by, for example, elements of a computing device. It will be recognized that various actions described herein can be performed by specific circuits (e.g., application specific integrated circuits (ASICs)), by program instructions being executed by one or more processors, or by a combination of both. Additionally, these sequence of actions described herein can be considered to be embodied entirely within any form of computer readable storage medium having stored therein a corresponding set of computer instructions that upon execution would cause an associated processor to perform the functionality described herein. Thus, the various aspects of the invention may be embodied in a number of different forms, all of which have been contemplated to be within the scope of the claimed subject matter. In addition, for each of the embodiments described herein, the corresponding form of any such embodiments may be described herein as, for example, “logic configured to” perform the described action.

The computer system may also include a main memory, such as a random access memory (RAM) or other dynamic storage device (e.g., dynamic RAM (DRAM), static RAM (SRAM), and synchronous DRAM (SDRAM)), coupled to the bus for storing information and instructions to be executed by processor. In addition, the main memory may be used for storing temporary variables or other intermediate information during the execution of instructions by the processor. The computer system may further include a read only memory (ROM) or other static storage device (e.g., programmable ROM (PROM), erasable PROM (EPROM), and electrically erasable PROM (EEPROM)) coupled to the bus for storing static information and instructions for the processor.

The computer system may also include a disk controller coupled to the bus to control one or more storage devices for storing information and instructions, such as a magnetic hard disk, and a removable media drive (e.g., floppy disk drive, read-only compact disc drive, read/write compact disc drive, compact disc jukebox, tape drive, and removable magneto-optical drive). The storage devices may be added to the computer system using an appropriate device interface (e.g., small computer system interface (SCSI), integrated device electronics (IDE), enhanced-IDE (E-IDE), direct memory access (DMA), or ultra-DMA).

The computer system may also include special purpose logic devices (e.g., application specific integrated circuits (ASICs)) or configurable logic devices (e.g., simple programmable logic devices (SPLDs), complex programmable logic devices (CPLDs), and field programmable gate arrays (FPGAs)).

The computer system may also include a display controller coupled to the bus to control a display, such as a cathode ray tube (CRT), liquid crystal display (LCD) or any other type of display, for displaying information to a computer user. The computer system may also include input devices, such as a keyboard and a pointing device, for interacting with a computer user and providing information to the processor. Additionally, a touch screen could be employed in conjunction with display. The pointing device, for example, may be a mouse, a trackball, or a pointing stick for communicating direction information and command selections to the processor and for controlling cursor movement on the display. In addition, a printer may provide printed listings of data stored and/or generated by the computer system.

The computer system performs a portion or all of the processing steps of the invention in response to the processor executing one or more sequences of one or more instructions contained in a memory, such as the main memory. Such instructions may be read into the main memory from another computer readable medium, such as a hard disk or a removable media drive. One or more processors in a multi-processing arrangement may also be employed to execute the sequences of instructions contained in main memory. In alternative embodiments, hard-wired circuitry may be used in place of or in combination with software instructions. Thus, embodiments are not limited to any specific combination of hardware circuitry and software.

As stated above, the computer system includes at least one computer readable medium or memory for holding instructions programmed according to the teachings of the invention and for containing data structures, tables, records, or other data described herein. Examples of computer readable media are compact discs, hard disks, floppy disks, tape, magneto-optical disks, PROMs (EPROM, EEPROM, flash EPROM), DRAM, SRAM, SDRAM, or any other magnetic medium, compact discs (e.g., CD-ROM), or any other optical medium, punch cards, paper tape, or other physical medium with patterns of holes, a carrier wave (described below), or any other medium from which a computer can read.

### V. Tangible Non-Transitory Computer Readable Storage Medium

Another aspect of the application relates to a tangible non-transitory computer readable storage medium, comprising instructions of a computer program that, when executed by a computer processor, cause the processor to: (a) receiving, via a user interface of an application executing on one or more computer processors, lung ultrasound images of a subject, wherein the lung ultrasound images are generated using a VSI protocol; (b) cropping, via the one or more computer processors, the gathered images to generate frames focusing on relevant lung areas; (c) segmenting, via the one or more computer processors, A-lines and other relevant structures from the frames; (d) quantifying, via the one or more computer processors, the number of A-lines in each frame to produce an average A-lines per frame (ALF); and (e) generating, via the one or more computer processors, an ultrasound scan result based on the ALF.

A computer program (also known as a program, software, software application, application, script, or code) can be written in any form of programming language, including compiled or interpreted languages, or declarative or procedural languages, and it can be deployed in any form, including as a standalone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

Additionally, the particular methods and/or corresponding acts in support of steps and corresponding functions described herein, may also be utilized to implement corresponding software structures and algorithms, and equivalents thereof. The processes described in this specification can be performed by one or more programmable processors (computing device processors) executing one or more computer applications or programs to perform functions by operating on input data and generating output.

Stored on any one or on a combination of computer readable media, the present invention includes software for controlling the computer system, for driving a device or devices for implementing the invention, and for enabling the computer system to interact with a human user. Such software may include, but is not limited to, device drivers, operating systems, development tools, and applications software. Such computer readable media further includes the computer program product of the present invention for performing all or a portion (if processing is distributed) of the processing performed in implementing the invention.

The computer code or software code of the present invention may be any interpretable or executable code mechanism, including but not limited to scripts, interpretable programs, dynamic link libraries (DLLs), Java classes, and complete executable programs. Moreover, parts of the processing of the present invention may be distributed for better performance, reliability, and/or cost.

Various forms of computer readable media may be involved in carrying out one or more sequences of one or more instructions to processor for execution. For example, the instructions may initially be carried on a magnetic disk of a remote computer. The remote computer can load the instructions for implementing all or a portion of the present invention remotely into a dynamic memory and send the instructions over the air (e.g., through a wireless cellular network or WiFi network). A modem local to the computer system may receive the data over the air and use an infrared transmitter to convert the data to an infrared signal. An infrared detector coupled to the bus can receive the data carried in the infrared signal and place the data on the bus. The bus carries the data to the main memory, from which the processor retrieves and executes the instructions. The instructions received by the main memory may optionally be stored on storage device either before or after execution by processor.

The computer system also includes a communication interface coupled to the bus. The communication interface provides a two-way data communication coupling to a network link that is connected to, for example, a local area network (LAN), or to another communications network such as the Internet. For example, the communication interface may be a network interface card to attach to any packet switched LAN. As another example, the communication interface may be an asymmetrical digital subscriber line (ADSL) card, an integrated services digital network (ISDN) card or a modem to provide a data communication connection to a corresponding type of communications line. Wireless links may also be implemented. In any such implementation, the communication interface sends and receives electrical, electromagnetic or optical signals that carry digital data streams representing various types of information.

The network link typically provides data communication to the cloud through one or more networks to other data devices. For example, the network link may provide a connection to another computer or remotely located presentation device through a local network (e.g., a LAN) or through equipment operated by a service provider, which provides communication services through a communications network. In preferred embodiments, the local network and the communications network preferably use electrical, electromagnetic, or optical signals that carry digital data streams. The signals through the various networks and the signals on the network link and through the communication interface, which carry the digital data to and from the computer system, are exemplary forms of carrier waves transporting the information. The computer system can transmit and receive data, including program code, through the network(s) and, the network link and the communication interface. Moreover, the network link may provide a connection through a LAN to a client device or client device such as a personal digital assistant (PDA), laptop computer, tablet computer, smartphone, or cellular telephone. The LAN communications network and the other communications networks such as cellular wireless and wifi networks may use electrical, electromagnetic or optical signals that carry digital data streams. The processor system can transmit notifications and receive data, including program code, through the network(s), the network link and the communication interface.

An aspect of the present application relates to a method of analyzing ultrasound images with an image processing system, comprising the steps of: gathering lung ultrasound images of a subject using a VSI protocol; cropping, by the image processing program, the gathered images to generate frames focusing on relevant lung areas; segmenting, by the image processing program, A-lines and other relevant structures from the frames; and quantifying, by the image processing program, the number of A-lines in in each frame.

In some embodiments, the image processing program further produces an average A-lines per frame (ALF) and generates an ultrasound scan result based on the ALF.

In some embodiments, the image processing program is an AI-assisted image processing program.

In some embodiments, the method further comprises the step of reviewing, by a trained expert, the ultrasound result generated by the image processing program.

In some embodiments, the segmenting step is performed by the image processing program using a segmentation algorithm.

In some embodiments, the quantification step is performed by the image processing program using an A-line counter algorithm.

In some embodiments, the gathering step is performed by a non-physician health personnel.

In some embodiments, the lung ultrasound images of a subject are sagittal images.

In some embodiments, the lung ultrasound images of a subject are transverse images.

In some embodiments, the ultrasound scan result is either abnormal or normal.

In some embodiments, an ALF equal to or less than 0.6 is identified as abnormal and an ALF above 0.7 is identified as normal.

In some embodiments, the method further comprises a step of using reinforcement learning to enable the image processing program to adapt to new data based on input on segmentations, wherein clinicians provide input on segmentations.

An aspect of the present application relates to a method of training an image processing program, comprising the steps of: (a) acquiring lung ultrasound images of a subject, wherein the lung ultrasound images are generated using a volume sweep imaging (VSI) protocol; (b) cropping the acquired lung ultrasound images to generate frames focusing on relevant lung areas; (c) segmenting, by a human medical professional, A-lines and relevant structures in the cropped lung ultrasound images to produce manual segmentations as ground truth; (d) assigning red-green-blue (RGB) colors to the ground truth to produce ground truth masks; (e) selecting frames with masks to focus on informative images for analysis; (f) applying a segmentation algorithm to the acquired lung ultrasound images to replicate the manual segmentations; and (g) repeating steps (a)-(f) until a desired hit-and-miss ratio (HMIR) is achieved.

In some embodiments, step (f) further comprises applying a counting algorithm to calculate average A-lines per frame (ALF) and produces a scan result based on ALF of the lung ultrasound images of a subject.

In some embodiments, the segmentation algorithm, counting algorithm, or both are AI algorithms.

In some embodiments, the lung ultrasound images of a subject are sagittal images.

In some embodiments, the ultrasound scan result is either abnormal or normal.

In some embodiments, an ALF equal to or less than 0.6 is identified as abnormal and an ALF above 0.7 is identified as normal.

An aspect of the present application relates to an image processing system, comprising: one or more computer processors; and one or more tangible computer readable media accessible by the one or more computer processors, wherein the one or more tangible computer readable media comprise instructions that, when executed by the one or more processors, cause the one or more processors to perform: (a) receiving, via a user interface of an application executing on one or more computer processors, lung ultrasound images of a subject, wherein the lung ultrasound images are generated using a VSI protocol; (b) cropping, via the one or more computer processors, the gathered images to generate frames focusing on relevant lung areas; and (c) segmenting, via the one or more computer processors, A-lines and other relevant structures from the frames.

In some embodiments, the one or more processors further perform the steps of: (d) quantifying, via the one or more computer processors, the number of A-lines in each frame to produce an average A-lines per frame (ALF); and (e) generating, via the one or more computer processors, an ultrasound scan result based on the ALF.

In some embodiments, the image processing system is an AI-assisted image processing system.

In some embodiments, the image processing system further comprises one or more client devices.

In some embodiments, the system is a cloud based system.

An aspect of the present application relates to a tangible non-transitory computer readable storage medium, comprising instructions that, when executed by a computer processor, cause the processor to: (a) receiving, via a user interface of an application executing on one or more computer processors, lung ultrasound images of a subject, wherein the lung ultrasound images are generated using a VSI protocol; (b) cropping, via the one or more computer processors, the gathered images to generate frames focusing on relevant lung areas; and (c) segmenting, via the one or more computer processors, A-lines and other relevant structures from the frames.

In some embodiments, the one or more processors further perform the steps of: (d) quantifying, via the one or more computer processors, the number of A-lines in each frame to produce an average A-lines per frame (ALF); and (e) generating, via the one or more computer processors, an ultrasound scan result based on the ALF.

In some embodiments, the tangible non-transitory computer readable storage medium is located on a client device.

In some embodiments, the tangible non-transitory computer readable storage medium is accessible through the cloud.

The present application is further illustrated by the following examples that should not be construed as limiting. The contents of all references, patents, and published patent applications cited throughout this application, as well as the Figures and Tables, are incorporated herein by reference.

### EXAMPLES

### Example 1

This study developed an automated segmentation and quantification system for A-lines in lung ultrasound images, leveraging the Attention U-Net architecture to differentiate normal from abnormal lung conditions. The system provides scalable screening and/or diagnostic solutions, using A-line counts to enhance access to pulmonary diagnostics in underserved regions/populations, outpatient settings and triage in health campaigns or hospitals/health care facilities. The system allows local or remote analysis of abnormal or borderline cases by a trained practitioner.

FIG. 1 shows VSI protocol applied to lung ultrasound.

FIG. 2 shows automatic segmentation and quantification of A-lines system workflow.

### Methodology

### A. VSI-Lung Dataset Collection

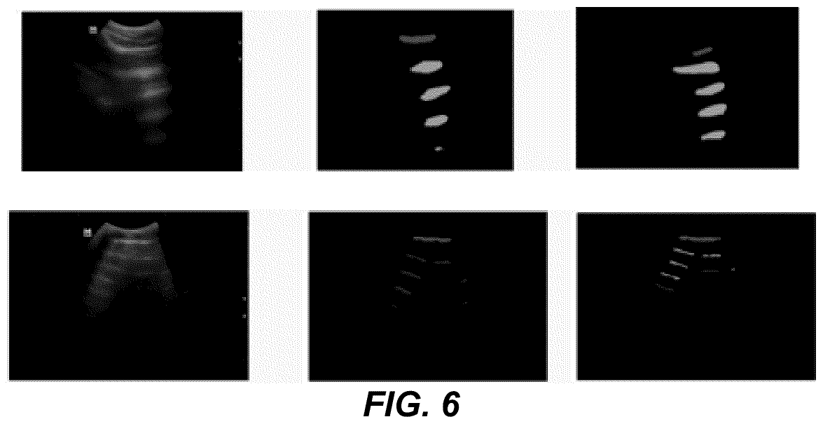

Gathered lung ultrasound images using the VSI protocol (FIG. 6) with standardized anatomical landmarks from Pasco, Peru.

### B. Pre-Processing (Cropping)

Cropped images to focus on relevant lung areas, improving clarity.

### C. Manual Segmentation

Designed and used Matlab's Meliora app for initial segmentation of A-lines and relevant structures. Three classes were defined: Pleural line, strong A-line and weak A-line.

### D. Post-Processing (RGB Colors Assignment)

Assigned RGB colors to ground truth for consistent identification. Red: Pleural line, Green: strong A-line.

### E. Frame Selection

Selected frames with masks to focus on informative images for analysis.

### F. Automatic Segmentation

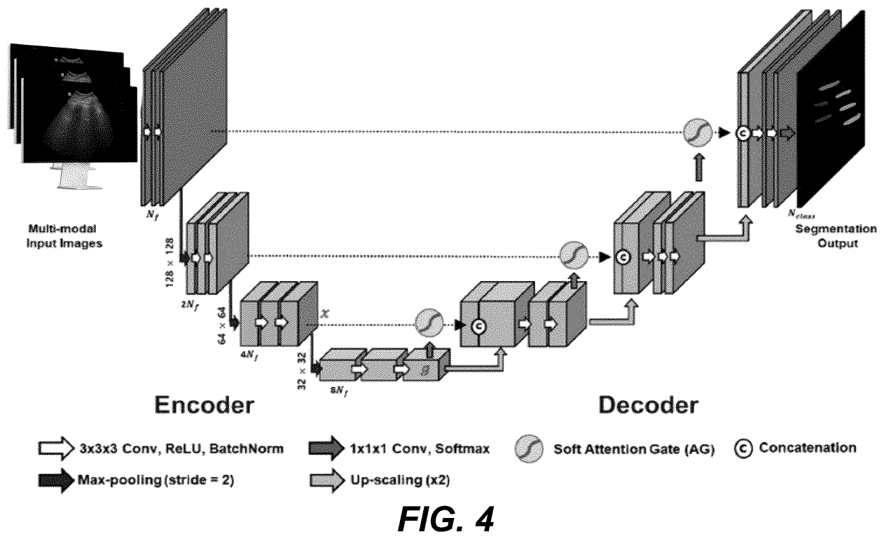

Applied an algorithm to replicate manual segmentation for scalability. Training was performed with 96 patients, using sagittal clips due to homogeneity of the data and baseline performance of the Attention U-Net architecture (FIG. 4) in comparison to transverse clips (Oktay O, Schlemper J, Le Folgoc L, Lee M, Heinrich M, Misawa K, Mori K, McDonagh S, Hammerla N Y, Kainz B, Glocker B, Rueckert D. Attention U-Net: Learning Where to Look for the Pancreas. arXiv. 2018 Apr. 11 [revised 2018 May 20]. Available from: https://arxiv.org/abs/i804.03999).

### G. A-Line Counting

Implemented an algorithm to count A-lines, distinguishing between normal and abnormal scans.

### Results

### Automatic Segmentation

FIG. 6 shows automatic segmentation of two frames for different patients.

### Relevant Metrics

Table 1 shows pixel-level performance of the Attention U-Net that produces the masks (pleura and A-lines) Recall, Dice Similarity Coefficient, Precision, and Accuracy evaluate the Deep Learning segmentation itself.

TABLE 1

| Type (test set) | Value (%) |
|---|---|
| Recall | 85 |
| Dice coefficient | 73 |
| Precision | 72 |
| Accuracy | 98 |

Table 2 shows performance of counting metrics from the connected-components A-line counter that runs after segmentation to turn masks into object counts per frame.

TABLE 2

| Type (100 frames test set) | Value (%) |
|---|---|
| Accuracy (prediction) | 99 |
| Accuracy(ground truth) | 99 |
| Precision | 100 |

### Statistical Analysis

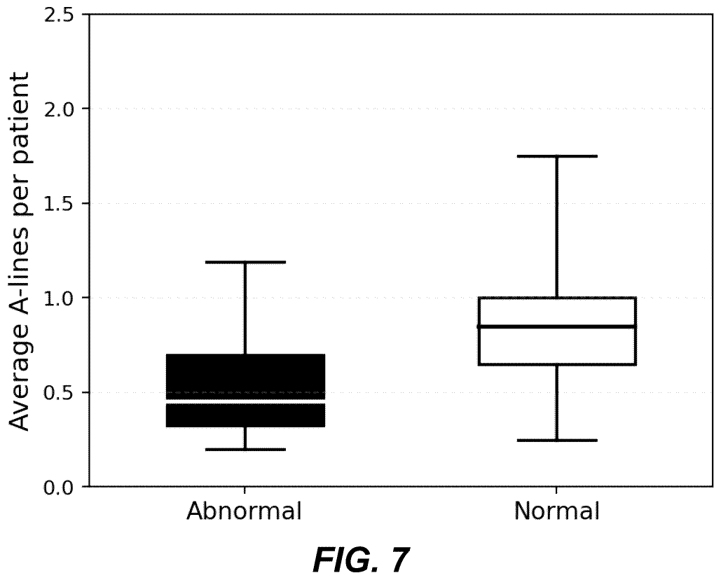

FIG. 7 shows a boxplot of relative A-lines per frame according to abnormal diagnosis.

### Example 2

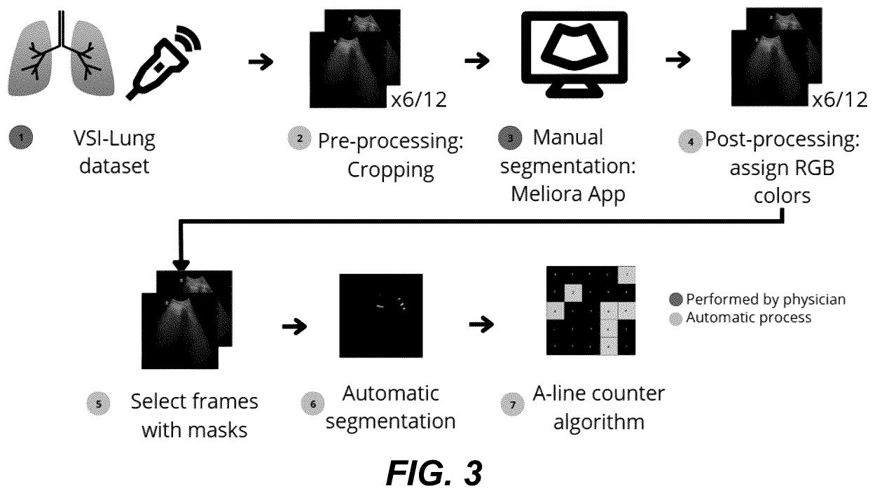

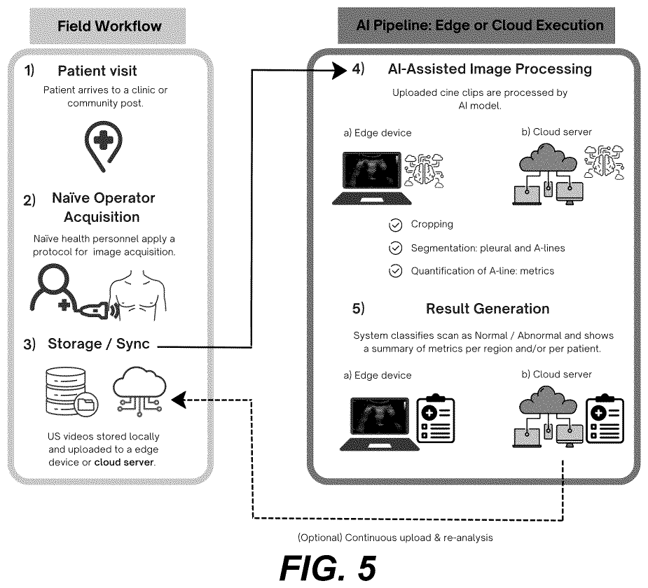

A proposed usage workflow is shown in FIG. 2, the algorithmic training workflow is shown in FIG. 3, and the deployment options (edge or cloud) for inference are shown in FIG. 5. Taken together, these figures describe: (i) point-of-care acquisition by non-physician personnel, (ii) storage/synchronization of cine clips, (iii) AI-assisted image processing that crops, segments, and quantifies A-lines, and (iv) generation of patient-level outputs and metrics for clinical interpretation or remote review. The detailed description for each step is as follows:

### A. Data and Acquisition

Lung VSI studies from 113 studies (98 normal, 15 abnormal) collected in Pasco, Peru (rural) and a second dataset from Rochester, New York, USA (urban) with 130 studies (103 normal, 27 abnormal). Studies were acquired using point of care ultrasound scanners.

### B. Annotation of Database for Training

Ground-truth masks were produced with a MATLAB application (M.E.L.I.O.R.A.) by expert radiologists. Classes included pleural line, strong A-line, and weak A-line.

### C. Preprocessing

Removal of on-screen metadata was applied with a cropping window on the ultrasound images, and selected frames with non-empty masks were extracted to focus on informative samples.

### D. AI Algorithm

U-Net, Attention U-Net and Multi-Attention U-Net models with Adam optimizer were trained on sagittal clips due to homogeneity and visualization advantages over transverse views. A k-fold cross validation of five splits was used for the Peru dataset, with a test set containing 20% of the normal studies and all abnormal studies. All Rochester dataset studies were used as a second test set.

### E. Post-Processing and Counting

Connected components for neighbor analysis with 8-connectivity for pixel and region discrimination was applied. From the predicted masks, we computed an A-line counting algorithm per frame to obtain comparison metrics between normal and abnormal studies. The following metrics were used:

1) Hit-and-miss ratio (HMR)—Each ground-truth A-line in a frame k was compared with all predicted masks to determine whether it was correctly detected. A hit was defined when there was any spatial overlap between a ground-truth region G and a predicted region P. The number of hits per frame was calculated as:

$${H}_{k} = {\sum }_{i = 1}^{{g}_{k}} 1 ( \vert {G}_{i}^{k} ⋂ {P}_{j}^{k} \vert > 0 ) \qquad ( 1 )$$

Where gₖ is the total number of ground-truth A-lines in frame k, and 1 is the indicator function that returns 1 if the condition is true.

HMR metric quantifies the relative difference between the number of A-lines detected by the model (Hₖ) and those annotated in the ground truth (Gₖ):

$$HMR = \frac{{H}_{k} - {G}_{k}}{{G}_{k}} \qquad ( 2 )$$

HMR is a counting-agreement metric that complements pixel-overlap measures such as Dice/IoU: while Dice evaluates segmentation overlap, HMR quantifies the relative over- or under-count of detected A-lines with respect to ground truth. Values close to zero indicate good agreement; negative values denote under-detection (misses) and positive values indicate over-detection (false positives). For reference, an aggregate HMR=0.72 (per Eq. (2)) means the algorithm over-counts by ˜72% on average.

2) Frame-level match accuracy: A frame-level accuracy metric was computed to measure agreement in the presence or absence of segmentations between prediction and ground truth. A frame was considered a match if both contained at least one A-line segmentation:

$$Frame match = {\sum }_{i = 1}^{N} 1 ( \sum {P}_{i} > 0 ∧ \sum {G}_{i} > 0 ) \qquad ( 3 )$$

3) Average A-lines per frame (ALF): ALF was calculated as the mean count of detected A-lines across all frames per sweep in each study:

$$A L F = \frac{1}{N} {\sum }_{i = 1}^{N} {a}_{i} \qquad ( 4 )$$

Where aᵢ is the number of A-lines identified in frame i, and N is the total number of frames. Group-level averages were then computed for normal and abnormal patients to compare aeration patterns.

### Statistics

Comparison between groups was made using a Mann-Whitney U test to assess significance in difference between normal and abnormal studies.

### Results

Using 113 studies (98 normal, 15 abnormal) from Peru (rural) and 130 studies (103 normal, 27 abnormal) in Rochester, USA (urban), an Attention U-Net architecture achieved robust pleural line and A-line segmentation. A-line counts per frame were significantly higher in normal than in abnormal scans (p<0.001), with averages of 2.55 vs 1.47 A-lines per frame, respectively.

The model achieved 0.81 Dice score segmentation on pleural lines and 0.63 on A-lines, respectively, in cross-site testing (Peru and Rochester). Relevant overall metrics are found in Table 3, with a 72% overall Hit and Miss ratio. Predicted A-line counts were ≈1.72×the ground-truth count. Frame-level match accuracy achieved 99% for both abnormal and normal patients. FIG. 6 shows a comparison between B-mode images, ground-truth segmentations and AI label predictions in two subjects from Peru.

TABLE 3
Overall Segmentation and Discrimination Metrics

| Metric | Value |
|---|---|
| Dice (pleural line) | 0.81 |
| Dice (A-lines) | 0.63 |
| HMR | 0.72 |
| Frame match | 0.99 |

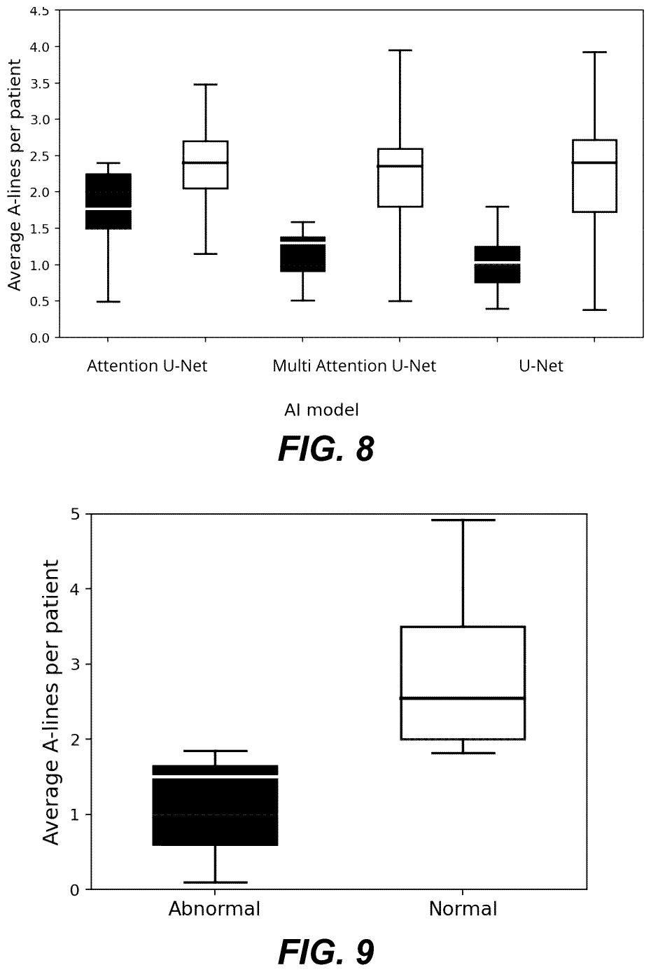

FIG. 8 shows a boxplot comparison of Average a-lines per patient between groups and according to different AI models in studies from Peru. Healthy patients presented a higher mean A-line count (2.26) compared to those with lung abnormalities (1.49), consistent across all models. Among the evaluated architectures, the Multi Attention U-Net achieved the largest inter-group separation in predicted A-line counts and exhibited lower variability across patients, suggesting improved sensitivity to structural lung changes. Based on this superior discrimination performance, the Multi Attention U-Net model was selected for the subsequent validation using the Rochester dataset. In that cohort, A-line counts remained significantly different between groups (p<0.001), averaging 2.55 for normal and 1.47 for abnormal studies (FIG. 9).

The strong cross-site performance between datasets acquired in Peru and Rochester demonstrates the feasibility of applying the proposed algorithm in remote low-resource settings with non-expert operators, using different ultrasound scanners, and in different populations. Although this version of the model was trained on a limited dataset, performance was achieved with relatively low information density, suggesting that results could be further improved with larger and more heterogeneous data.

The ability of the model to generalize across handheld ultrasound devices indicates that deep learning-based segmentation can be integrated into low-cost, portable platforms to facilitate field deployment. These results support AI-assisted screening using a VSI workflow that could help refer patients to healthcare centers with a higher resolutive capacity.

### Example 3

This application conducts a comprehensive analysis of A-line patterns in normal vs. abnormal lung studies from a bigger dataset.

Reinforcement Learning for Real-time Segmentation: Implements a feedback system using reinforcement learning, where clinicians provide input on segmentations, allowing the model to adapt.

Generalization for Multiple Ultrasound Devices: Adapts the model to function across different ultrasound equipment.

While various embodiments have been described above, it should be understood that such disclosures have been presented by way of example only and are not limiting. Thus, the breadth and scope of the subject compositions and methods should not be limited by any of the above-described exemplary embodiments but should be defined only in accordance with the following claims and their equivalents.

The above description is for the purpose of teaching the person of ordinary skill in the art how to practice the present invention, and it is not intended to detail all those obvious modifications and variations of it which will become apparent to the skilled worker upon reading the description. It is intended, however, that all such obvious modifications and variations be included within the scope of the present invention, which is defined by the following claims. The claims are intended to cover the components and steps in any sequence that is effective in meeting the objectives there intended, unless the context specifically indicates the contrary.

What is claimed is:

1. A method of analyzing ultrasound images with an image processing system, comprising the steps of:
gathering lung ultrasound images of a subject using a VSI protocol;
cropping, by the image processing program, the gathered images to generate frames focusing on relevant lung areas;
segmenting, by the image processing program, A-lines and other relevant structures from the frames; and
quantifying, by the image processing program, the number of A-lines in in each frame.

2. The method of claim 1, wherein the image processing program further produces an average A-lines per frame (ALF) and generates an ultrasound scan result based on the ALF.

3. The method of claim 1, wherein the image processing program is an AI-assisted image processing program.

4. The method of claim 1, further comprising the step of reviewing, by a trained expert, the ultrasound result generated by the image processing program.

5. The method of claim 1, wherein the segmenting step is performed by the image processing program using a segmentation algorithm.

6. The method of claim 1, wherein the quantification step is performed by the image processing program using an A-line counter algorithm.

7. The method of claim 1, wherein the gathering step is performed by a non-physician health personnel.

8. The method of claim 2, wherein the ultrasound scan result is either abnormal or normal.

9. The method of claim 8, wherein an ALF equal to or less than 0.6 is identified as abnormal and an ALF above 0.7 is identified as normal.

10. The method of claim 1, wherein the method further comprises a step of using reinforcement learning to enable the image processing program to adapt to new data based on input on segmentations, wherein clinicians provide input on segmentations.

11. A method of training an image processing program, comprising the steps of:
(a) acquiring lung ultrasound images of a subject, wherein the lung ultrasound images are generated using a volume sweep imaging (VSI) protocol;
(b) cropping the acquired lung ultrasound images to generate frames focusing on relevant lung areas;
(c) segmenting, by a human medical professional, A-lines and relevant structures in the cropped lung ultrasound images to produce manual segmentations as ground truth;
(d) assigning red-green-blue (RGB) colors to the ground truth to produce ground truth masks;
(e) selecting frames with masks to focus on informative images for analysis;
(f) applying a segmentation algorithm to the acquired lung ultrasound images to replicate the manual segmentations; and
(g) repeating steps (a)-(f) until a desired hit-and-miss ratio (HMIR) is achieved.

12. The method of claim 11, wherein step (f) further comprises applying a counting algorithm to calculate average A-lines per frame (ALF) and produces a scan result based on ALF of the lung ultrasound images of a subject.

13. The method of claim 12, wherein the segmentation algorithm, counting algorithm, or both are AI algorithms.

14. A tangible non-transitory computer readable storage medium, comprising instructions that, when executed by a computer processor, cause the processor to:
(a) receiving, via a user interface of an application executing on one or more computer processors, lung ultrasound images of a subject, wherein the lung ultrasound images are generated using a VSI protocol;
(b) cropping, via the one or more computer processors, the gathered images to generate frames focusing on relevant lung areas; and
(c) segmenting, via the one or more computer processors, A-lines and other relevant structures from the frames.

15. The tangible non-transitory computer readable storage medium of claim 14, wherein the tangible non-transitory computer readable storage medium further comprises instructions that, when executed by a computer processor, cause the processor to:
(d) quantifying, via the one or more computer processors, the number of A-lines in each frame to produce an average A-lines per frame (ALF); and
(e) generating, via the one or more computer processors, an ultrasound scan result based on the ALF.

16. The tangible non-transitory computer readable storage medium of claim 14, wherein the tangible non-transitory computer readable storage medium is located on a client device.

17. The tangible non-transitory computer readable storage medium of claim 14, wherein the tangible non-transitory computer readable storage medium is accessible through the cloud.

18. An image processing system, comprising:
one or more computer processors; and
tangible non-transitory computer readable storage medium of claim 14.

19. The image processing system of claim 18, wherein the image processing system is an AI-assisted image processing system.

20. The image processing system of claim 18, wherein the system is a cloud based system.
